(19) United States
(12) Patent Application Publication
(10) Pub. No.: US 2013/0053701 A1
(43) Pub. Date: Feb. 28, 2013

(54) DERMATOSCOPE AND ELEVATION MEASURING TOOL

(71) Applicants: Ingo Wiest, Berlin (DE); Joern Ole Becker, Berlin (DE)

(72) Inventors: Ingo Wiest, Berlin (DE); Joern Ole Becker, Berlin (DE)

(73) Assignee: W.O.M. WORLD OF MEDICINE AG, Berlin (DE)

(21) Appl. No.: 13/497,498

(22) PCT Filed: Sep. 24, 2010
(86) PCT No.: PCT/EP10/64165

§ 371 Date: Apr. 18, 2012

(30) Foreign Application Priority Data
Sep. 24, 2009 (DE) 10 2009 044 962.0

(51) Int. Cl. A61B 1/04; A61B 1/00; A61B 1/06

(52) U.S. Cl. 600/476; 600/109; 600/104

(57) ABSTRACT

A dermatoscope is in the form of a handheld device is provided. The dermatoscope comprising a display which is arranged in or on a housing, for an image of the skin surface and/or of a structure of the skin surface which is recorded by a surface sensor of the dermatoscope, and a storing means for storing the images recorded by the dermatoscope.

### CROSS-REFERENCE TO A RELATED APPLICATION

This application is a National Phase Patent Application of International Patent Application Number PCT/EP2010/064165, filed on Sep. 24, 2010, which claims priority of German Patent Application Number 10 2009 044 962.0, filed on Sep. 24, 2009.

### BACKGROUND

The present invention relates to a dermatoscope and an elevation measuring instrument.

The correct visual primary diagnosis of a pigmented or non-pigmented precancerous or cancerous skin lesion by a medical specialist for dermatology is typical between 60 to 80%. An improvement can be obtained additionally when looking at the skin with the aid of a dermatoscope and/or a video dermatoscope system (additional integration of an image sensor for displaying the image on a computer monitor).

The dermatoscopy is a non-invasive method for the in vivo assessment of non-pigmented and pigmented skin lesions. The use of the epiluminescent technique allows an assessment of structures just being beneath the skin surface by eliminating reflections on the skin surface, what also provides a considerable improvement in the differential diagnostics. The epiluminescence is mostly obtained by optical coupling via a cover glass and a fluid (immersion oil or disinfection spray), more rarely by using polarized light and cross polarization filter in the display optics. The handling of the mentioned fluids is usually considered in practice as disturbing (contamination, in particular when using immersion oil, time loss by applying the fluid, extra costs due to the expendable items, in particular in case of immersion oil instead of for instance disinfection spray).

Incident light dermatoscopes comprise usually a fixed enlargement (typical 10 times) for measuring lesions. Glass discs with printed measurement scales can often be inserted into the optical path. The position of the measurement scale is thereby not variable.

The use of conventional dermatoscopes requires that the examining doctor has to look at the screening area through an ocular and thus a physical small working distance between the examining person and the patient is the unavoidable consequence. In particular, when examining the genital zone the privacy of the patient is not guaranteed. For both the patient as well as for the examiner the individual distance is considerably fallen below, which is the reason that the examination is considered by both parties as latent uncomfortable. Furthermore, the ergonomics for the examiner is evaluated as not being optimal. The handling is also hindered due to the often chunky construction and relatively high weight corresponding therewith. A diagnostic documentation is in a lot of cases possible by adapting digital cameras; however a remodelling of the dermatoscope is required for each change between diagnosis and documentation. Handling, size, weight, losable parts and so on are strongly negatively affected.

When using diagnosis supporting systems currently available on the market, as video dermatoscopy systems, the protection of the privacy is also violated by the apparatus immanent requirement for imposing the image recording head on the skin surface, however due to the decoupling of observation point (at a monitor) and recording point (at the patient) a considerably higher individual distance is achieved. In case of video dermatoscopy systems always only a stationary measurement is possible, since these devices are cable-bound. A majority of these systems offers a diagnosis support based on mathematical algorithms for assessing the malignancy, wherein in particular the so called “ABCD rule” obtained a high prominence and practicability: Due to a systematically, objective and standardized recording and evaluation of the criteria symmetry, boundary, colour scheme (colouring) and differential structure the differentiation between a good-natured pigmentation disorder and for instance a malignant melanoma shall be improved.

### SUMMARY

It is the object to develop means which offer more information to the examiner in an efficient and very easily handable manner.

The dermatoscope according to an exemplary embodiment of the invention comprises the form of a handheld device, wherein a display which is arranged in or on a housing, for an image of the skin surface and/or a structure of the skin surface is provided, wherein the image was recorded by the dermatoscope. Furthermore, the dermatoscope comprises a storing means for storing the images recorded by the dermatoscope.

According to a further exemplary embodiment of the invention an elevation measuring instrument is provided, wherein the detection of structures which are vertical to the skin surface and/or in the skin is possible.

BRIEF DESCRIPTION OF THE DRAWINGS

Embodiments are illustrated in following exemplarily by the means of Figures.

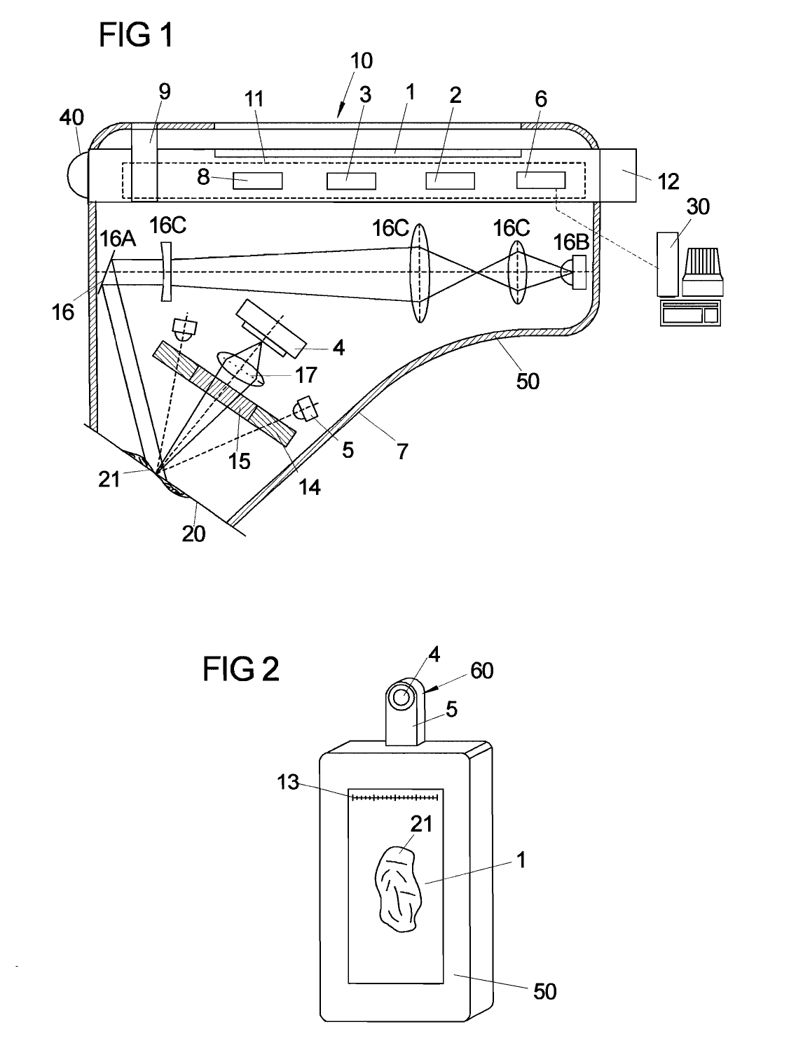

FIG. 1 shows the principle construction of an embodiment of a dermatoscope when detecting a structure on a skin surface and an elevation measurement.

FIG. 2 shows a schematic perspective view of an embodiment of a dermatoscope.

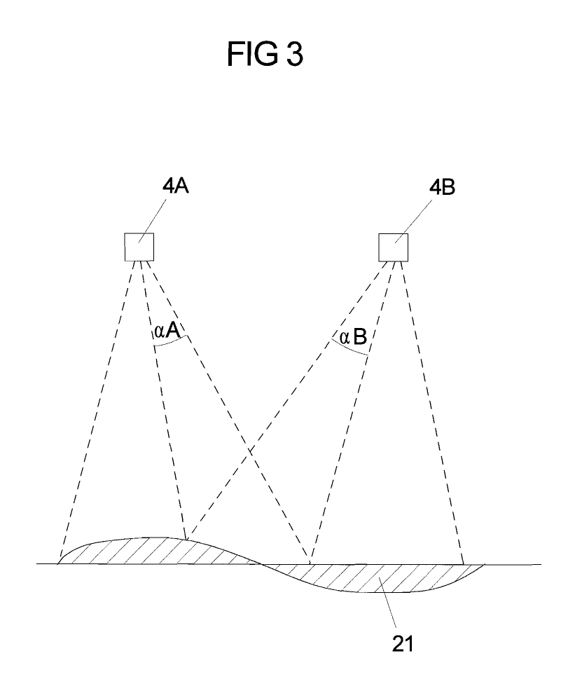

FIG. 3 shows a schematic illustration of an embodiment for elevation measurement.

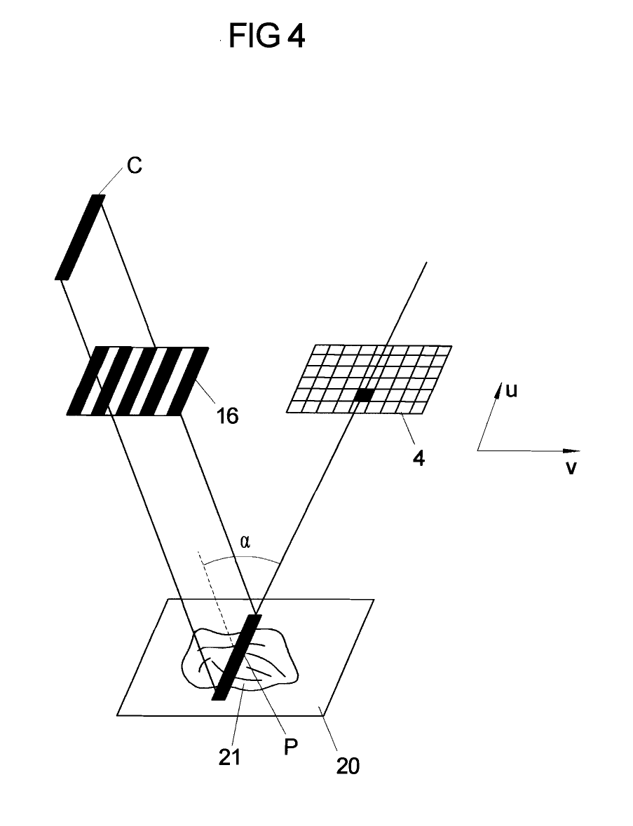

FIG. 4 shows a schematic illustration of an embodiment of an elevation detection.

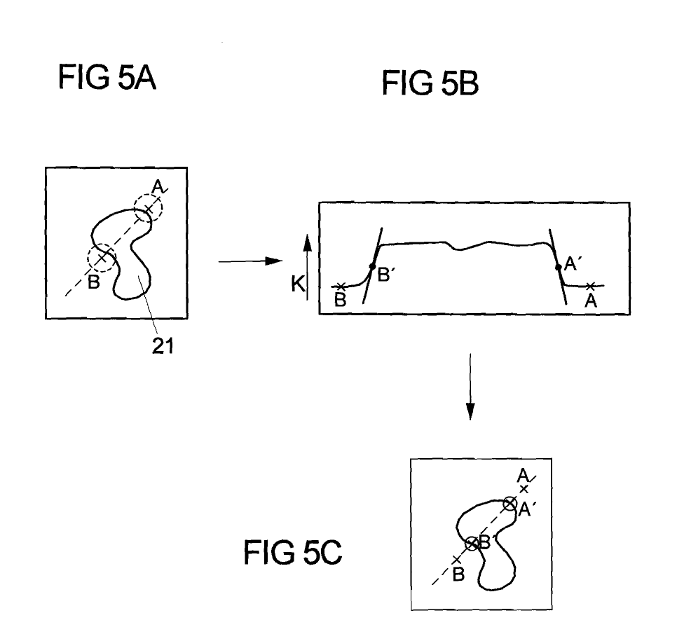

FIGS. 5A-C show schematic illustrations of a measurement of a structure on a skin surface.

### DETAILED DESCRIPTION

Before referring to the different applications of the dermatoscope and the elevation measuring instrument at first different embodiments of the dermatoscope 10 are described. Thereby, it is obvious for the person skilled in the art that the combination of features in the embodiments illustrated herein are only exemplarily and also other combinations of the described features are possible. This also encloses the omission of singular features.

In FIG. 1 an embodiment of a dermatoscope 10 is illustrated in a schematic manner in a sectional view from the side. The dermatoscope 10 comprises a housing 50, which is here represented only schematically. The housing 50 comprises curved form areas which allow for a simple and save handing. Furthermore, such a designed housing can be easily cleaned or disinfected.

A skin surface 20 is illustrated below the dermatoscope 10, on which structures 21 are located, which shall be examined by the means of the dermatoscope 10. A skin surface 20 is here and in the following understood to be not only the surface of the skin in the narrow medical sense with epidermis, dermis and hypodermis, but also mucose membranes (tunica mucosa) which coat for instance mouth, nose or the rectum. As will be explained in the following in more detail embodiments of the dermatoscope 10 can also cover areas coated with a mucose membrane such that the term of the dermatoscope 10 is expanded.

In FIG. 1 the skin surface 20 and the structure 21 are illustrated in a sectional view, wherein it is deducible that the structure 21 comprises an irregular form vertical to the skin surface. The structure 21 can be elevated above the skin surface 20, but can also be lowered in respect to the skin surface 20. The structure 21 in FIG. 1 shows both features.

The dermatoscope 10 is designed as a mobile handheld device. The basic body can have about the size of a smart phone (for instance length 150 mm, with 62 mm, depth 12 mm). The spacer 7 is then arranged at the basic body.

The dermatoscope 10 is also usable autonomously like a smart phone that means it is not connected constantly to a power supply or a computer. Thus, the dermatoscope 10 can be for instance carried during a visit, wherein patients can continuously be examined using the dermatoscope 10 during the visit.

As will be explained later, other embodiments of the dermatoscope 10 can also comprise interfaces for the current supply and data transfer.

Size specifications and shape specifications for the dermatoscope 10 are only to be understood as being exemplary. They show that this dermatoscope 10 can be used as a handheld device by the doctor comfortably during the examination without that for instance cable impede the required movements during work.

An energy storing device 8 for energy supply is provided in and/or the dermatoscope 10, which should provide a sufficient capacity for about 1 working day (at least 5 hours operating time). Due to the favourable weight/capacity ratio lithium polymer accumulators should be used, wherein also other kinds of energy supply are conceivable (as for instance other accumulator types, condensers for energy storage and so on).

The charging of an integrated (an ideally replaceable in analogy to a mobile telephone) energy storing device 8 (for instance accu) can occur typically by plugging in a basis station (docking station, cradle), by connecting to a recharger cable or in a specifically preferred variant of this invention by applying an induction coil, wherein in the interior of the dermatoscope 10 also a coil for inductive energy coupling is provided.

Such a dermatoscope 10 can be used for determination, evaluation, documentation and follow-up of skin diseases, in particular skin cancer. These skin diseases have in common that they provide structures 21 which are the basis for diagnosis.

The dermatoscope 10 comprises a data processing system 11, which controls the different components of the dermatoscope 10, monitors and/stores data. In the following some of the components of the dermatoscope 10 are described, wherein different embodiments of the dermatoscope 10 can also comprise different combinations of the components.

For recording structures 21 on the skin surface 20, the dermatoscope comprises a surface sensor 4, with which the skin surface 20 can be scanned. Thereby it is reasonable, if the optical surface sensor 4 is coupled to an optical system 14, 15, 17 (for instance an enlarged lens system, polarisers, see FIG. 4), which images an image area with a diagonal about 10 to 30 mm (preferably 20 to 25 mm) on the surface sensor 4. These dimensions are reasonable for a lot of examinations, wherein naturally also other image area dimensions are possible.

When starting from a sensor in the format ½, 5″ diagonal (˜10 mm) and a 4:3 format, the image area comprises in the object plane a diagonal of 25 mm and the display comprises a diagonal of 4.5″ in 4:3 format, this corresponds to a total magnification by a 1:1 illustration on the display of about 4.5×. In case of an image area of 10 mm a magnification of about 12× would be achieved, in case of an image area of 13 mm a magnification of about 4× would be achieved.

In order to receive good measurement results it is reasonable to provide a surface sensor 4 with at least two megapixels. CMOS-sensors and/or CCD sensors integrated into the housing 50 of the dermatoscope 10 can for instance serve as surface sensors 4. Alternatively, also a monochromatic surface sensor 4 can be used.

The colour image can be calculated by selective illumination using a variation of the illumination colours.

In order to obtain reproducible results during the examination, the dermatoscope 10 can comprise a spacer 7. In the embodiment illustrated in FIG. 1 the spacer 7 is designed in form of a protrusion of the housing 50. The protrusion as spacer 7 can also be designed in an embodiment as half shell. Furthermore, it is alternatively or additionally possible to use a contact-free spacer 7, in case of which it is indicated to the user of the dermatoscope 10 for instance by optical, haptical (for instance vibrations) and/or acoustic means if a pre-adjusted or calculated distance is maintained or not. It can be reasonable that an elevation measurement is carried out in a contact-free process, an incident light recording on the other side using a stationary spacer 7.

Furthermore, a pattern projector 16 illustrated in FIG. 1 is illustrated with multiple exemplary components. The function of the pattern projector 16 is being explained in more detail with reference to FIG. 4. The pattern projector 16 comprises a light source 16B, which can be provided as an LED. The light is guided through a lens system 16C to a light control device 16A (for instance a digital light processor or also a micro mirror array) from which the light is guided to the structure 21.

In any case this dermatoscope 10 does not require a support glass which touches the skin surface 20 of the patient. Therewith it is also guaranteed that no device immanent artefacts would be generated by pressing the skin structures.

Furthermore, the exemplarily illustrated dermatoscope 10 comprises an illumination means 5, with which the skin area to be monitored can be illuminated in an even and in a predetermined manner.

In the illustrated embodiment the illumination means 5 comprises a plurality of LEDs, which are arranged on the circumference (for instance annular) of the optical system and/or on the circumference of the surface sensor 4. The brightness and/or the colour selection of the illumination means 5 can be varied. In an alternative embodiment the pattern projector 16 and the illumination means 5 can be integrated into one component.

The skin area to be microscoped and to be monitored in respect to the elevation (see below for a detailed description) is evenly illuminated during the examination by the illumination 5. In particular the use of white light LED as well as any combination of white light LED with LED of other spectral ranges, in particular in the blue/near UV range and/or infrared range are possible. When using a black-white surface sensor 4 it can be reasonable for instance to control blue and green LEDs one after the other in order to be able to calculate a colour image from the different illumination.

It is also possible that the skin area to be examined can be radiated sequentially and/or parallel in different spectral ranges. If an image is taken in each of the different illumination types, then the comparison of the images can provide valuable additional information for the diagnosis.

The optical system 14, 15, 17 comprises in the embodiment illustrated in FIG. 1 a polarization system with a cross polarising filter 14, 15, which is arranged in the beam path between the skin surface 20 and the surface sensor 4. An object of the polarization system is to reduce reflections of the skin surface 20 as much as possible. The cross polarizer comprises two portions 14, 15, wherein the first portion 14 forms the polarizer for the illumination, the second portion 15 forms the polarizer for the surface sensor 4. The polarization directions of the two portions 14, 15 are selected such that the one of the second portion 15 is shifted by 90° to the first portion 14. Furthermore, the optical system comprises a lens system 17, which is only schematically illustrated in FIG. 1.

By using the mentioned polarization technique the application of contact fluids can be avoided, what provides considerable advantages in the application, as mentioned above. This technique serves also for assessment of underlying morphological structures by almost eliminating reflections on the skin surface.

With the aid of the optical systems 14, 15, 17, the illumination means 5 and the surface sensor 4 images can be taken and can be stored in the dermatoscope 10. The dermatoscope 10 comprises therefore a storing means 2, which is part of a data processing means 11 or is coupled thereto.

By integrating the storing means 2 into the dermatoscope 10 the temporary storage of the recorded images of the screening area is possible. The storage size is thereby designed such that the screenings of a complete working day can be stored. The storing media can thereby be provided for instance as an integrated memory (transient or non-transient memory types for instance RAM, hard disc etc.) or/and in combination with an extendable or/and replaceable memory (for instance SD, mini SD memory chip, flash memory, USB memory stick and so on). All stored data can be provided automatically with a time labelling, a patient identification (ID) and/or a signature of the examiner.

The patient identification can be obtained in a specifically simple manner, if the dermatoscope 10 comprises a reading means 9 for a code. The reading means 9 can for instance scan a barcode from a patient file so that it is guaranteed that all recorded or calculated data in the dermatoscope 10 are assigned clearly to the correct patient. An alternative or additional possible embodiment comprises photographing a code or similar information and subsequent decoding by an image analysis.

The embodiment according to FIG. 1 comprises additionally a camera system 40 (for instance a digital camera module with auto focus and flash light), with which recordings can be taken with a conventional camera as for instance a mobile camera. The recordings of the camera system 40 can be stored in the same storing means 2. Due to the additional integrated camera system 40 the need is cancelled to provide an additional camera for other recording situations.

Thus, the dermatoscope 10 offers the possibility to provide via the camera/video unit of the camera system 40 an overview image (for instance microscopic recordings) for assigning a detailed recording. The camera system 40 allows also recording of a larger skin area or of the whole patient in order to document the localization of a detailed recording as well as of a microscopic recording.

Alternatively and/or in addition thereto the retrieval of a suspicious skin lesion (that means a structure 21) can be facilitated by illustrating a schematic scheme of the human body on the display 1, in which the position of the suspicious lesion can be added. In order to be able to make a suitable selection the input of the location can be realized via a touch screen directly on the display 1 in a particular preferred variant of this invention. In an alternative, a handling via a mouse wheel, track ball etc. is conceivable.

In order to obtain a higher level of detail an area of the body can be selected as for instance an arm, which subsequently can be again illustrated as schematic scheme format fitting on the display 1. After successive detailing the touch screen allows subsequently the marking of the measurement location. The illustration on the body scheme besides detail scheme and marking of the measurement location on the same scheme occurs for each single lesion.

It is also reasonable, if the dermatoscope 10 comprises a storing means 2 for language data, such that the examiner can comment during the examination, which is then also automatically assignable in a timely manner and to the patient personally. The language data can be provided in a data format as for instance a wave format (*.wav) or an mp3 format (*.mp3). The dermatoscope 10 can also comprise a corresponding coding algorithm, in order to convert *.wav data to *.mp3 data. Furthermore, the dermatoscope 10 can comprise means for language recognition and can produce text messages from *.wav as well as *.mp3 data and can store them on the storing means 2.

Recorded images, stored images and/or other information can be illustrated on an integrated display 1. Since the dermatoscope 10 shall be portable, it is reasonable to adapt the size of the display 1. If for instance a touch screen (touch sensitive display) is used, then an image diagonal of 3:6 inch, preferably 3.5:5.5 inch can be used. The display 1 can be designed for instance as an integrated display with an LCD, TFT or OLED.

The application of a touch screen has the advantage that the same means for display can be used for entering information. Alternatively or additionally, a keyboard can be provided on the dermatoscope 10 for entering data input. It can furthermore be helpful, if a mouse wheel and/or a track ball are arranged on the housing 50 of the dermatoscope 10. Therewith, for instance a cursor can be guided on the display 1.

In FIG. 1 the display 1 is integrated directly into the housing 50 of the dermatoscope 10. In other embodiments the display 1 can also be connected hinged to the dermatoscope 10 such that the Examiner can easily change the view angle to the display 1 without changing the orientation of the dermatoscope 10 in respect to the skin surface 21.

Usually, the structures 21 to be examined are surrounded by hair and/or are overgrown by hair. Thus, it can be important, before recording by the dermatoscope 10 to remove the hair. In order to elevate this, the dermatoscope 10 can comprise an electrical shaving means 12, which is for instance completely integrated into the housing 50 when not used.

If required, the shaving means 12 can be pulled out of the housing 50. In FIG. 1 the shaving means 12 is visible in a pulled out position. The shaving means is arranged in a small area of the dermatoscope 10, so that a shaving is also possible at narrow locations. In case of a suitable arrangement of the shaving means 12 the shaving can also be followed by video observation on the display 1. Thus, it is guaranteed, that the shaving occurs gentile that means that in particular none of the structures 21 is injured during the shaving.

If an examination of a skin area is now carried out, then it is reasonable to illustrate the signal of the surface sensor 4 as a relative low (in monitor resolution) resolved video stream in real time on the display 1 for directing the optical system 14, 15, 17 towards the measurement location to be examined. Therewith, the examiner can look at the skin surface 20 in a relaxed working posture in order to be able eventually to take subsequently a recording and here eventually to be able to examine singular areas in an enlarged manner. Thus, it is not any longer necessary for the examiner to move with the eyes into close proximity of a dermatoscope 10 and thus to the examined body.

If then images are being taken, which optionally shall be stored in the storing means 2, then it is reasonable to illustrate these images as images with high resolution on an integrated display in a variable enlargement.

In case of the low resolved (monitor resolution) as well as each high resolved illustration of the images (any enlargement up to the native resolution of the sensor) it is reasonable, if automatically and/or optionally a scale 13 (for instance linear measure) is superimposed into the image, which can additionally also be stored. When varying the enlargement scale of the picture on the display 1 the scaling as well as the enlargement scale can be changed in analogy to the resolution.

The data processing device 11 comprises a means with which the contour and/or the size of a structure 21 on and/or in the skin surface 20 can be detected. As soon as the structure 21 is detected the enlargement of the image on the display is selected such that the structure is visible in a good manner, for instance centrally and in the largest possible illustration on the display 1. Distance, area and diameter measurements can also be taken on the image, for instance by marking said points on the image of the display 1 between which the measurement has to occur. The data processing device 11 analyzes the points or traverses, in order to calculate a value there from. An example is described in reference to FIG. 5. These measurements can also be used in order to automatically find a (variable) enlargement scale preferred by the observer so that the examining object is illustrated format fitting.

It is for instance possible to store in addition to the native image also the image supplemented by the measurement points and/or measurement scale and/or enlargement scale.

For a further processing of the recorded and/or calculated data, in particular the images, the dermatoscope 10 can comprise at least one interface 6 to an external computer system 30 or another diagnostic system.

Possible interfaces 6, which can also be used in combination, are for instance a GPRS-data connection, a WLAN-interface, a ZigBee-interface, a USB-interface, a Bluetooth-interface. In general, the at least one interface 6 can be provided wireless or also cable-bound.

Thereby, it is in general possible to send data (for instance image and/or audio data) in both directions. Thus, it is possible to upload previous recordings of a skin location onto the dermatoscope 10 and to compare them to a present recording.

On external computer systems 30 for instance an expanded follow-up is possible or image analytic and image database requests in a histological safeguarded expert system can be carried out with a further processing unit. The mobile dermatoscope 10 can also be supported during the analytics by building up a wireless or cable-bound communication between the external computer system 30 and the dermatoscope 10. Hereby, it can be guaranteed by suitable database structures and encryption routines that the recorded and stored data (for instance image and elevation data) are assigned clearly to the patent, the recordings have been taken of.

The external computer system 30 can be connected to an additional monitor, which can be seen as reasonable within the frame of the patient communication, in particular in the separate area.

In FIG. 2 a perspective view of a further embodiment of a dermatoscope 10 is illustrated, as explained with reference to FIG. 1; the above description can be thus referenced. The housing 50 of the dermatoscope 10 is essentially rectangular, wherein the display 1 is arranged on one of the flat sides. A structure 21 is schematically illustrated on the display, which has been recorded by the surface sensor 4. A scale 13 is superimposed in the display for a better diagnosis, wherein said scale is automatically scaled when adjusting the enlargement.

In FIG. 2 the surface sensor 4 is not arranged on one of the flat sides, but on a small bar 60 as an endoscopic element on the front face of the housing 50; the actual recording direction of the skin surface would occur in FIG. 2 into the image plane. Due to the small bar (for instance width smaller than 1 cm) the surface sensor can also be introduced into skin pockets or other narrow body areas. The endoscopic element 60 can also comprise specific shapes which can be adapted for instance for the exploration of body orifices. It can comprise the shape of a laryngoscope for the examination of the throat. Thereby, the endoscopic elements 60 can be arranged stationary or movably (for instance as goose neck) on the housing 50. It is basically also possible to design the endoscopic element 60 in a removable manner.

After describing embodiments of the dermatoscope 10, an application will be described in the following, which basically can also be used independently on the dermatoscope 10, namely the detection of elevation data (that means data relating to the rising or lowering of a structure 21 relative to the skin surface 20) of the structures 21. Elevation data comprise the extension of the structure 21 in vertical direction, which means vertical to the skin surface 20.

It is basically known to determine and evaluate the shape contour of structures 21 such as moulds. In the following, the extension in vertical direction (so called elevation) is also detected and evaluated. Finally, it is possible to determine a volume of the structure 21.

The data relating to the contour, the elevation and/or the volume can be stored like the recorded images.

In case of a suitable embodiment a dermatoscope 10 is thus realizable which can be combined with an elevation measuring means. Herewith, it is possible to obtain microscopically high resolved recordings of the skin surface 21 and to measure the skin topography with one or multiple spatial sensors in a time-synchronic manner via a suitable three-dimensional measurement. An adapted registering process allows hereby the assignment of the image to the space information.

Methods for measuring surfaces are integrated into the beam path of the imaging optics for elevation measurement. The methods of the laser triangulation, photogrametry and interferometry are here mentioned exemplarily. In case of the method of photogrametry, in particular the relevant close-up photogrametry, three-dimensional structures are determined by taking recordings of the structures in two or more directions (or positions). This is illustrated with reference to FIG. 3. A possible embodiment would be if, for instance two or more surface sensors 4A, 4B are arranged spatially distributed on or in the housing 50, which record a structure 21 from different view angles (α_{A}, α_{B}) (see FIG. 3).

This can also be achieved alternatively or additionally in that different regions of a surface sensor 4 are headed for in order to create different view angles. Finally, the calculation of the spatial structure 21 is based on triangulations.

Alternatively or additionally, laser scanner, structured light (for instance randomized structures) or encoded light can be used for scanning the structure 21.

An embodiment seems to be in particular of an advantage using the techniques of the structured light, thus the projection of an active structured stripe illumination on the skin surface (see FIG. 4). An encoding of the illumination (for instance by means of grey code, phase shifting and/or colour coding) is hereby of particular advantage, wherein said illumination is projected from the pattern projector 19 to the skin surface 20 and the structure 21. The surface sensor 4 is in particular advantageously operated for the elevation measurement simultaneously as stripe projection sensor.

In FIG. 4 it is illustrated that the projector 16 illuminates in particular the structure 21 with patterns of parallel bright and dark stripes of different width. A time sequence of different brightness values is obtained for each image point of the surface sensor 4.

The desired three-dimensional coordinates of the surface can be calculated in two steps:

The pattern projector 16 corresponds thereby to a reversed camera. The number C of the stripes can be calculated from the sequence of encoded brightness values, which are measured from the image sequence for each camera image point. The stripe number C specifies hereby a light plane in space.

Besides, in case of a provided object point P on the structure of the skin surface 21 the image coordinates u, v in the image of the surface sensor 4 are also known. This image coordinate corresponds to a light beam (thus a straight line) between the object point P and the image coordinate on the flat sensor 4.

Since the position between the flat sensor 4 and pattern projector 16 is constant, a constant triangulation basis is provided here. Simultaneously, the flat sensor 4 registers the projected stripe pattern in a known, constant view angle to the projection a. The intersection of the plane and the straight line can subsequently be calculated; herewith the desired three-dimensional coordinate of the object point in the coordinate system of the sensor can be determined.

The data processing device 11 controls the signal output of the pattern projector 16 and/or the signal input by the surface sensor 4 and evaluates the signals reflected and/or scattered by the skin surface 20 or the structure 21 and determines therewith the elevation of the structure 21. This can be illustrated in a superimposed manner, for instance in form of elevation line images on the display 1 (or a screen on a host computer) of the image of the skin 20.

The elevation determination can be carried out in a device, which is independent on the described dermatoscope. The illustrated embodiments show however combined embodiments.

Following the description of the dermatoscopic photographs and the elevation measurement the processing of the data is described subsequently, wherein the data have to be understood as being primarily the image data and/or the elevation data.

The image and measurement information (in particular also the elevation data) can be analyzed with the help of a local (this means a system arranged within the dermatoscope 10), decentral or central evaluation system for the computer assisted diagnosis and can be illustrated on a display 1 serving the diagnosis on the apparatus and/or an external monitor. The evaluation system is hereby designed for instance on the basis of a histological secured image database and allows thus an automatic comparison of the current and previous recordings of a lesion with suitable “similar” reference diagnoses. This can in particular be carried out, when the dermatoscope 10 is connected to a host computer.

This integrated interactive image atlas offers the doctor a further diagnosis support in form of a second opinion as well as a transparent and reconstructable basis for the decision finding. A suitable encoded system for storage is provided for the storage of the data.

Besides the already previously mentioned ABCD criteria the evaluation of the elevation of a lesion (this means a structure 21) offers a further diagnostical decision basis: While a non-malignant lesion has a slow growth, a fast three-dimensional size growth is recognizable in case of malignant melanoma. Rough and shedding lesions, which bulge outwards, are hereby considered as being basically suspicious and can point to a melanoma. Although elevated lesions can of course easily be palpated, thus the requirement of an elevation measurement is obviously not given, it is to be considered that in particular the change (the “E” of the ABCD rule stands besides elevation also for evolution, thus development) as velocity measurement of growth/change is decisive for diagnostic analysis.

Besides increase also a decrease of the elevation extension serves as indicator, especially since regressions phenomena in pigment lesions are known to be more expression of a potential deterioration of the character of the tumour. With regard to the light skin cancer a more differential evaluation is necessary.

It is obvious that the quantitative follow-up of the elevation is not possible by palpation (touch), also the lesion is flattened by the cover glass of the dermatoscope typically present in the epiluminscence diagnostics independent on using or not using oil or liquid emersion. With regard to the dimension of the elevation an artefact is thus basically provided. In the course of the follow-up examinations other examiners become part of the assessment process and assessments are moved. The typical photo documentation does also not provide sufficient security to changes with regard to the elevation and relative relations to changes others as for instance ABCD parameters. An inclusion of “E” in a multivariant analysis of ABCD is completely missing so far. With regard to the ABCD rule when detecting pigmented lesions and the broad differential diagnostics as well as the use in case of light skin cancer the dermatoscopy is thus inferior in respect to the third dimension, namely the elevation, detection by the eye of the examiner and palpitation.

By using the dermatoscope 10 with integrated elevation measurement technique it is in particular possible to combine the three dimensions “visual observation” and “palpitation” of elevations and “microscopical enlargement” for providing a diagnosis, to determine the elevation herefore metrological and to set in statistical ratio the continuation of the elevation in particular within the frame of skin cancer examination for malignant melanoma in respect to constancy, decrease or/and increase, irregularities of the surface in respect to constancy or increase or decrease in the respective course parallel to the classical ABCD parameters or other evaluation algorithms.

With reference to FIG. 5 an embodiment is described, in which an image recording (with or without elevation measurement) can be used to determine the lateral dimensions of a structure 21.

In FIG. 5A a structure 21 is illustrated which can be seen by a user on a display 1. The user can with a pointing means as for instance a cursor select two points A and B in the image. The points A, B are selected such that the structure 21 is between the points A, B.

Along the straight line continuing through the points A, B for instance the contrast change K is determined (for instance by the data processing device 11) by a threshold value process. The profile obtained thereby is illustrated in FIG. 5B. The turning points A′, B′ can now be numerically determined at the progress of the contrast change. The distance between the turning points A′, B′ is a measure for the size of the section through the structure 21 along the straight line.

The contrast gradient is for instance illustrated in FIG. 5B. The edge of the structure 21 is determined by the turning points A′, B′ in the contrast gradient. Alternatively or additionally to the contrast change also other threshold value methods (for instance evaluation of the brown percentage, gamma-value or the colour situation) can be used.

In FIG. 5C it is illustrated how the distance between the points A′, B′ and thus an expansion of the structure 21 along the straight line through these points can be determined.

If this process is conducted several times, the extension of the two-dimensional structure 21 can be well determined and documented.

It is in particular of an advantage to measure time-synchronously to the generation of the microscopically high resolved recordings of the skin surface 20 the skin topography with one or multiple spatial sensors using a suitable measurement method. It is further of an advantage, to provide an adapted registry method in order to safeguard the assignment of the image information to the space information. Using suitable measures as mentioned above, it is thus possible to omit a cover glass such that the skin elevation is not being deformed by overlaying the dermatoscope 10 onto the skin, thus artefacts being imminent to the apparatus can be avoided.

The image and measurement information can be realized with the help of a local, decentral or central evaluation system for the computer assisted diagnosis. The local evaluation system can be coupled to the data processing device 11 of the dermatoscope.

The evaluation system contains a calculation means for evaluating the ABCD rule based on a histological secured image database. The evaluation means contains further an automatic comparison of the present recording or previous recordings of the same lesion with suitable reference diagnoses identified as being similar within the frame of mathematical algorithms, as this is for instance known from DE 0000 102 39 801 A1, DE 10 2004 003 329 B3, DE 10 220 423 A1.

An interactive image atlas integrated into the evaluation system can offer the examiner an extended diagnosis support in way of a second opinion as well as a transparent and reconstructable basis for decision making.

Such in particular wireless dermatoscope 10 comprises an integrated documentation function. Hereby, it can be excluded that the patient has to go to the documentation facility, that the dermatoscope 10 has to be connected with the aid of an adapter to a camera for documentation purposes or that the patient has to be sent to a separate documentation employee in a hospital, which photographs the lesions previously identified by the doctor as being conspicuous and enters said photographs into a hospital network. Through this, additional cost in the clinic and loss of time can be avoided.

Classical optical imaging dermatoscopes comprise typically a stationary (10 times) enlargement. A size estimation of skin structures is in most cases possible via stationary scales introduced into the optical beam path. The dermatoscope 10, on the other side, offers variable enlargements, wherein a positionable scale or a measurement function is adapted to the respective enlargement.

Due to the technical combination of image recording and elevation measurement, wherein the measurements can be conducted time-synchronously or sequentially, a multimodal sensor unit is provided. In a particular preferred embodiment of the invention both sensor types are referenced to each other.

For the purpose of a timely follow up of suspicious skin areas there is in particular the possibility to display an image of a skin area recorded at an earlier time point as reference image. It can be switched back and forth between both images (the present and the reference image or images). This allows it in particular preferably to recognize changes of the suspicious lesion.

This is in particular advantageously solved if when updating a so called working list (listing of the patients to be presently examined) the patient data of all following patients including all present or stored images relating to the person are loaded via an interface 6 from an external computer 30 to the dermatoscope 10. When selecting the comparative function an automatic segmentation occurs of the present image as well as of the reference image, for instance in reference to the method of threshold value recognition described in FIG. 5. After segmentation an algorithm tries to bring the pictures into congruency as much as possible.

A segmentation is here to be understood that a contourline of the structure 21 is assembled of singular points, for instance the location of the strongest change of the colour situation or highest contrast change. A polygon path is created with which the contour of the structure 21 can be approximated with a pre-settable accuracy.

When recording a structure, in particular in a timely distance, it can occur that the segmentations of the structure 21 do not completely succeed and a congruent superimposition is hereby not possible. Possible reasons for mistakes are for instance different view angles, a changed skin, the different selection of segmentation and any artefacts (as for instance hairs).

However, in order to allow a comparison, in particular an automatic comparison of structures 21 with reference images, it can be reasonable that the Examiner selects in both images at least one distinctive point (for instance a specific characteristic shape of the structure 21, a scar, etc.) such that a subsequent automated assignment of the present image is alleviated by rotation and side shift of the image to the reference image, wherein the algorithm strives primary for a superimposition of the previously selected distinctive points. A distortion or a size variation of the image is not to be provided since herewith an undesired image manipulation would be accompanied.

A back and forth switching between the images is now possible.

An image algorithm downstream of the recording allows a simultaneous evaluation of multiple image information recorded one after the other or together (white light image, elevation image/data set plus optionally further images) in real time by using multimodal colour texture approaches, which allow to characterize and to classify spatial and spectral percentages of a lesion simultaneously. Furthermore, known features can be integrated smoothly into the evaluation system based on the ABCD rule. Hereby new multimodal algorithms for characterizing skin lesions are provided.

Thereby, it is of an advantage to realize this image algorithm besides ABCD (E) rules via an image based similarity evaluation in comparison to histological secured reference diagnoses. The algorithm suggests the user histological secured references diagnoses on the integrated and/or external display parallel or alternating to the presently recorded lesion and offers the Examiner hereby a further aid to decision making for the own diagnosis. The histological secured reference data set can thus be understood and used within the meaning of an interactive dermatological atlas. Thereby, the two-dimensional images can also be processed as multimodal data sets.

Independent of a complex mathematical and/or image based evaluation algorithm, which can be optimized for running time optimization by shifting to performant separated system, it is in particular of an advantage to realize a system, which comprises additionally a relative simple image evaluation unit on the mobile dermatoscope 10, which examines by the means of electronic pattern recognition the respective screening field in respect to the presence of pigment marks. A pigment mark is to be understood in this reference as a continuous skin area, which differs significantly in respect to its pigmentation from the remaining skin area. A detected pigment mark can automatically be enlarged illustrated via this electronic pattern recognition such that it fills out optimal the available area of the integrated display. Equally, a detected pigment mark can be automatically measured regarding its size (for instance colour, structure, length, width, circumference). In an alternative manifestation or in addition thereto by marking a point on the display the distance between these points can be calculated by the mobile dermatoscope 10 and can be illustrated on the display; the image supplemented by these measurement points, a measurement scale and image/enlargement scale can be stored additionally to the native image in respect to the lesions. In particular, when determining the diameter of a lesion by using this measurement function by the means of a touch panel without input aid means, rather via finger, it is conceivable and in particular preferred to implement suitable image processing algorithms for edge recognition (for instance by the means of contrast recognition bright/dark) such that the demarcation of the lesion occurs automatically near to the pressure point on the touch panel and thus said point is preselected, which has the largest contrast difference near the pressure point (see picture 5).

It is provided to vary the enlargement of the illustrated screening area. The specific advantage for the doctor is that certain areas of suspicious pigment marks can be illustrated in a particular high enlargement for elevating the diagnosis. Due to the very high image resolution of the image sensor in comparison to image resolution of the display no deterioration of the image quality is accompanied with an enlargement. The enlargement function is compatible to the above-mentioned measurement function such that the measurements can occur in respect to the resolution. The scaling (and the display of the enlargement factor) changes in analogy to the selected enlargement scale.

Furthermore, a connection to practice software systems by implementing a BDT/GDT-compatible interface is possible (treatment data transfer interface “BDT”, and apparatus transfer interface “GDT”) as well as a connection to a hospital information system (KIS) for instance via HL7 (Health Level 7) standard is possible. Also the connection to a PACS-system (Picture Archiving and Communication System) is appropriate by implementing a corresponding DICOM interface (Digital Imaging and Communications in Medicine).

It is in particular suitable to request the so called working list in DICOM standard (working list, order of the patient's to be examined on a medical apparatus) from the KIS system by the described server system and to transfer wireless or cable-bound to the mobile dermatoscope 10. Alternatively, it would also possible to transfer a working list, which is based on existing data of the medical practice (for instance by connecting to the local administration software), but not necessarily in the DICOM standard.

Hereby, it can be achieved that the patient list is automatically provided to the doctor; the images and elevation data can thus be assigned very simple to an existing patient data set. Additionally it is conceivable to illustrate further patient-related information from the KIS system on the mobile dermatoscope 10 via an integrated DICOM viewer.

Alternatively or additionally thereto the dermatoscope 10 comprises in a particular preferred embodiment a chip card reading device 9, with which for instance health insurance cards can be read. Hereby, in particular in the area of doctor's offices a simple detection/transfer of patient-related data for recording/elevation measurement can occur such that the received data can be accordingly assigned in the patient administration system. An alternative embodiment comprises a barcode reader. The scanner can be provided for instance as a separate laser scanner or as an integrated 2D data matrix scanner, which evaluates the image data of the image sensor via a suitable algorithm.

In the following, different aspects of a method are described by describing a dermatoscope 10 and/or an elevation detecting means 3.

Methods for examining the skin of a test person, during which by the means of a wireless, compact and mobile dermatoscope 10 a suspicious skin area (screening area) is optimally illuminated and illustrated on an integrated display for diagnosis.

Thereby, the camera signal can be illustrated as low resolved video stream in real time on the apparatus display 1 for aligning the dermatoscope 10 to the measurement location to be examined.

It is furthermore possible to record by the means of an optical surface sensor 4 after manual activation the screening area with high resolution.

The recorded screening area can also be stored in an internal storing means 2.

The recorded screening area can also be illustrated on the integrated display 1 in a variable enlargement.

Furthermore, the method comprises an electronic pattern recognition, which analyses the screening area in respect to the presence of pigment marks (structures 21) and enlarges them such that the integrated display 1 is filled out to a maximum.

It is possible that the recognized pigment marks are automatically measured.

Due to the pattern recognition recognized pigment marks can be analyzed in respect to their geometrical, structural and/or colour properties by suitable algorithms.

The elevation of suspicious skin locations (structures 21) can also be measured by a suitable technology.

For the purpose of the follow-up of suspicious skin areas images and/or elevation data of previous recordings/measurements of the identical skin areas as reference data can be shown on the display.

It is possible that based on an image-based similarity evaluation histological secured reference diagnoses are suggested to the doctor on the integrated and/or external display parallel or alternating to the presently recorded/measured lesion for comparison purposes.

Possibilities for recording voice messages are provided, which can be stored compressed or non-compressed.

In addition to detail recording of a lesion and/or a measurement of the elevation an overview recording of a larger area can be possible in order to be able to localize a detail measurement. The localization of an overview image, a detail recording and/or an elevation measurement is also possible which is simplified in that a schematic sketch of a human body is illustrated on the display, on which the position entry of the suspicious area to be examined can occur.

The recorded image data and/or audio data as well as the data about the elevation measurement or the data of the evaluation algorithm are transferred to a separated server system which can communicate with the examining system cable-bound or also wireless.

1. A dermatoscope in form of a handheld device comprising
a display which is arranged in or on a housing for an image of the skin surface and/or a structure of the skin surface, which is recorded by a surface sensor of the dermatoscope, and
storing means for storing the images recorded by the dermatoscope.

2. The dermatoscope according to claim 1, further comprising at least one elevation detecting means for detecting structures being vertical to the skin surface.

3. The dermatoscope according to claim 2, wherein the at least one elevation detecting means comprises means for photogrammetric detection of three-dimensional structures, a means for laser triangulation for detecting three-dimensional structures and/or a means for interferometry for detecting three-dimensional structures and/means for triangulation using an actively structured illumination.

4. The dermatoscope according to claim 2, wherein the elevation detecting means comprises at least two spatially separated surface sensors and/or spatially separated areas of an surface sensor for recording the structure at different angles.

5. The dermatoscope according to claim 2, by further comprising a data processing means with a computing means for automatic determination of the volume of the structure.

6. The dermatoscope according to claim 2, wherein the elevation detecting means is coupled to a light source for structural illumination.

7. The dermatoscope according to claim 1, further comprising a diagnostic system, the results thereof can be illustrated on the display.

8. The dermatoscope according to claim 1, further comprising at least one optical surface sensor, in particular by a SMOS sensor or a CCD sensor integrated into the housing, for image recording of a screening area.

9. The dermatoscope according to claim 1, wherein the at least one surface sensor for studying skin in skin wrinkles or openings is arranged on a projection of the housing, a bar on the housing of the dermatoscope and/or is designed as a rotatable and/or hinged surface sensor.

10. The dermatoscope according to at least one of the preceding claim 1, further comprising a spacer for keeping the distance between the surface sensor or the elevation detecting means and the skin surface constant.

11. The dermatoscope according to claim 1, further comprising an illumination means, in particular a concentrical ring illumination or an illumination at the circumference of the optical system, for an even illumination of the skin area to be measured and documented.

12. The dermatoscope according to claim 11, wherein the illumination means emits when operated white light, wherein the white light can be realized by a combination of LED with other spectral ranges.

13. The dermatoscope according to claim 12, wherein the combinations of LED comprise also spectral ranges in the blue/near UV range and/or infrared range.

14. The dermatoscope according to claim 11, wherein the skin area to be examined can be irradiated sequentially and/or parallel in different spectral ranges by the illumination means.

15. The dermatoscope according to at least one of the claim 11, wherein the light of the illumination means is guided through at least one first polarizing element, in particular a polarization filter, designed as a disc, film or plastic plate such that the light effecting the skin surface comprises a defined vibration level.

16. The dermatoscope according to claim 11, wherein a second polarizing element is arranged in front of the beam path of an image detector, which is arranged in orthogonal vibration direction relative to the first polarizing element.

17. The dermatoscope according to claim 1, wherein measuring functions, in particular a linear measurement, circumferential measurement and/or a volume measurement are integrated.

18. The dermatoscope according to claim 1, further comprising means for automatic creation of a linear scale for display and/or storage of an image.

19. The dermatoscope according to claim 1, wherein when marking two points on and/or in a recorded image the distance of both points can be calculated and can be displayed on the display and the currently selected imaging scale and/or enlargement scale and/or measurement scale can be displayed optionally analogue on the display.

20. The dermatoscope according to claim 1, further comprising an image processing means for edge detection of the structure in and/or on the skin surface, in particular a threshold value process as for instance contrast detection bright/dark.

21. The dermatoscope according to claim 19, wherein when selecting two points on a touch panel a demarcation of the lesion near the pressure points is automatically performed, thus said points are automatically preselected, which represent locally the largest threshold value difference and correspond with very high likelihood to points of the borderlines of a lesion.

22. The dermatoscope according to claim 1, further comprising an electronic pattern detection for analyzing the screening area for the presence of pigments.

23. The dermatoscope according to claim 22, wherein by using the pattern detection significant pigmentation differences can be automatically detected in dependency on if the found pigment can be automatically displayed on the display enlarged such that the area being available is optimally used and/or a centred illustration occurs.

24. The dermatoscope according to claim 1, further comprising a means for recording voice messages.

25. The dermatoscope according to claim 1, wherein the storing means comprises at least one port for a storing means, in particular for a RAM component, a hard disc, an SD card, a mini SD card, a flash memory and/or a USB memory stick.

26. The dermatoscope according to claim 1, further comprising an energy storage device, in particular, a battery, in particular a lithium polymer battery and/or a means for inductive energy coupling.

27. The dermatoscope according to claim 1, further comprising a visualization means for displaying data on the display, in particular elevation data of previous recordings/measurements as reference data for the follow-up measurement.

28. The dermatoscope according to claim 1, further comprising a means for segmentation of at least one structure.

29. The dermatoscope according to claim 28, wherein at least two segmentations of the structure can be automatically adapted to each other and/or an adaptation occurs by the means of at least one point selected by the user.

30. The dermatoscope according to claim 29, further comprising a means for switching back and forth and corresponding display between the current measurement and at least one reference measurement on the display and/or a previous reference measurement parallel on the display.

31. The dermatoscope according to at claim 1, further comprising at least one wire-based or wireless interface for data transfer.

32. The dermatoscope according to at claim 1, further comprising a means for sequential or simultaneous evaluation and transfer of multiple image information detected one after the other or together, in particular white light image, elevation data, optional further images with different spectral illumination in real time or at a later time point.

33. The dermatoscope according to claim 1, further comprising an evaluation means and/or diagnostic means for automatic evaluation and/or diagnosis of the obtained data, in particular based on the ABCD (E) rule.

34. The dermatoscopy according to claim 1, further comprising at least one reading means for a code, in particular a barcode and/or matrix code.

35. The dermatoscope according to claim 1, further comprising shaving means.

36. The dermatoscope according to claim 1, wherein the combination of techniques and methods for image recording and for elevation measurement forms a multi-modal sensor unit.

37. The dermatoscope according to claim 36, wherein both sensor types are referenced to each other.

38. An elevation measuring instrument for detecting of structures being vertical to the skin surface on and/or in the skin.

39. The elevation measuring instrument according to claim 38, wherein the at least one elevation detecting means comprises means for photogrammetric detection of three-dimensional structures, a means for laser triangulation, and/or a means for interferometry and/or means for triangulation using an actively structured lightening.
